(19) United States
(12) Patent Application Publication
(10) Pub. No.: US 2011/0240050 A1
(43) Pub. Date: Oct. 6, 2011

(54) COSMETIC COMPOSITIONS CONTAINING A FATTY ACID, ARGININE, AND A CO-EMULSIFIER

(75) Inventors: Gisela Perruna, Rahway, NJ (US); Dhaval Patel, Edison, NJ (US); Sara Muenz, Middlesex, NJ (US)

(73) Assignee: L'OREAL S.A., Paris (FR)

(21) Appl. No.: 13/078,468

(22) Filed: Apr. 1, 2011

Related U.S. Application Data
(60) Provisional application No. 61/320,072, filed on Apr. 1, 2010.

(51) Int. Cl. A61Q 1/02; A61K 8/92; A61Q 1/00

(52) U.S. Cl. 132/200; 514/558

(57) ABSTRACT

Disclosed are cosmetic compositions containing a fatty acid, arginine, and a co-emulsifier. Also disclosed are methods for making the cosmetic composition and applying it to keratinous tissue such as skin in order to enhance its appearance.

### CROSS-REFERENCE TO RELATED APPLICATIONS

This application claims the benefit of the filing date of U.S. Provisional Patent Application No. 61/320,072, filed Apr. 1, 2010, the disclosure of which is hereby incorporated herein by reference.

### BACKGROUND OF THE INVENTION

The present invention generally relates to a novel cosmetic composition and method of use, such as a foundation or eye shadow and method of making-up skin. More particularly, the present invention relates to foundation and eye shadow compositions which have good physical properties and long wear, which is achieved through the use of a combination of a fatty acid and arginine.

It is well known in the industry to neutralize fatty acids contained in cosmetic emulsions. Generally, the fatty acid is neutralized by a base to form a soap that can act as an emulsifier to aid in the emulsification of the water and oil phases. However, commonly used neutralizers suffer from significant drawbacks. For example, sodium hydroxide produces emulsions that are hard creams with poor sheen. Potassium hydroxide produces emulsions that are soft creams with good sheen, but a large amount of the neutralizer is required. Amides such as triethanolamine produce emulsions that have good sheen, but risk the production of nitrosamine byproducts, which are feared to be carcinogens. Ammonia produces emulsions with a strong, offensive odor, and ammonia itself is a harsh base, which can be dangerous to use in manufacture.

Therefore, it is desirable to provide cosmetic compositions, such as foundations and eye shadows, having excellent physical properties and sheen, which do not suffer from any of the drawbacks of conventional neutralizers.

### SUMMARY OF THE INVENTION

A first aspect of the present invention is directed to a cosmetic composition, including a foundation or eye shadow, containing a fatty acid, arginine, and a co-emulsifier.

A second aspect of the present invention is directed to a method of preparing a cosmetic composition, including a foundation or eye shadow, by mixing together a fatty acid, arginine, and a co-emulsifier.

A third aspect of the present invention is directed to a method of making up or enhancing the appearance of a keratinous tissue by applying to the keratinous tissue a cosmetic composition, including a foundation or eye shadow, containing a fatty acid, arginine, and a co-emulsifier.

It has been surprisingly discovered that a composition according to an embodiment of the present invention has excellent physical properties and good sheen, without any of the drawbacks of conventional neutralizers.

### DETAILED DESCRIPTION

Other than in the operating examples, or where otherwise indicated, all numbers expressing quantities of ingredients and/or reaction conditions are to be understood as being modified in all instances by the term “about,” meaning within 10% to 15% of the indicated number.

“Keratinous tissue”, as used herein, includes but is not limited to, skin, hair and nails.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen-containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C. “Non-volatile”, as used herein, means having a flash point of greater than about 100° C.

As used herein, the expressions “at least one”, “a”, and “an” mean one or more and thus include individual components as well as mixtures/combinations.

“Long wear” compositions as used herein, refer to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

### Fatty Acid

According to the present invention, compositions comprising a fatty acid are provided. The fatty acids useful in the present invention are any cosmetically or dermatologically acceptable and, in general, physiologically acceptable fatty acid. Examples of fatty acids include stearic acid, lauric acid, palmitic acid, arachidic acid, myristoleic acid, palmitoleic acid, sapienic acid, oleic acid, linoleic acid, arachidonic acid, eicosapentaenoic acid, erucic acid, and docosahexaenoic acid. In an embodiment of the present invention, the fatty acid is a C₁₂-C₁₆ fatty acid. In an embodiment of the present invention, the fatty acid is stearic acid.

The fatty acid is present in the inventive compositions in amounts generally ranging from about 0.1% to about 15%, in some embodiments from about 0.5% to about 10%, in some embodiments from about 1% to about 5%, by weight, and in yet other embodiments from about 1.5% to about 2%, by weight, based on the total weight of the cosmetic composition.

### Arginine

According to the present invention, compositions comprising argentine are provided. Arginine is a semi-essential amino acid. Typically, arginine is found in the L-form (commercially available from Ajinomoto), which is bioavailable to mammals. In an embodiment of the present invention the arginine is a natural or synthetic L-arginine. Arginine is present in the inventive compositions in amounts generally ranging from about 0.01% to about 5%, in some embodiments from about 0.05% to about 2%, in some embodiments from about 0.1% to about 1%, by weight, based on the total weight of the cosmetic composition. In embodiments of the present invention arginine is present in an amount of about 0.2%, about 0.67%, or about 0.90%, by weight, based on the total weight of the composition.

When the fatty acid and the arginine are combined, the arginine neutralizes the fatty acid to form an emulsifier (or soap). Thus, the compositions of the present invention include both a fatty acid and arginine in a combination that promotes and/or maintains an emulsion.

According to the present invention, compositions comprising a co-emulsifier are provided. The co-emulsifier aids the soap formed by the combination of the fatty acid and the arginine in promoting and/or maintaining the emulsion. The co-emulsifiers useful in the present invention are any cosmetically or dermatologically acceptable and, in general, physiologically acceptable substances, other than the combination of the fatty acid and arginine, that promote or maintain an emulsion.

The co-emulsifiers useful in the present invention include amphoteric/zwitterionic surfactants, nonionic surfactants, cationic surfactants, and anionic surfactants.

Representative examples of suitable amphoteric surfactants include lauryl betaine, lauroamphoglycinate, lauroamphopropylsulfonate, lauroamphopropionate, lauroamphocarboxyglycinate, lauryl sultane, myristamidopropyl betaine, myristyl betaine, myristoamphoglycinate, myristyl propionate, stearoamphoglycinate, stearoamphopropionate, stearoamphopropylsulfonate, stearyl betaine, cocamidoethyl betaine, cocamidopropyl betaine, cocamidopropyl hydroxysultane, cocamidopropyl dimethylamine propionate, cocoamphoglycinate, cocoamphocarboxypropionate, cocoamphocarboxyglycinate, coco-betaine, cocoamphopropionate, and cocoamphopropylsulfonate.

Representative examples of suitable nonionic surfactants include fatty alcohols, such as cetyl alcohol, fatty acid esters, and alkoxylated, particularly ethoxylated, fatty acid esters of polyhydric alcohols such as glycerols and sorbitol, for example, polyoxyethylene monolaurate, polyoxyethylene monooleate, polyoxyethylene monostearate, sorbitan monolaurate, sorbitan trioleate, glyceryl stearate, steareth-12, ceteth-10, and PEG-100 stearate, generally with a degree of ethoxylation of from about 20 to about 85; mono- and di-alkanolamides, such as the N-acyl derivatives of mono- and di-ethanol amines, and polyethoxylated monoalkanolamides; amine oxides, such as cocoamidopropyl dimethylamine oxides, coco bis-2-hydroxyethyl amine oxides and lauryl dimmethylamine oxide; ethoxylated alkanolamides; ethoxylated oils and fats such as ethoxylated lanolins; ethoxylated alkylphenols, such as Nonoxynol, and polaxamers.

Representative examples of suitable anionic surfactants include the following: sodium stearoyl lactylate, the alkali metal, ammonium, or amine salts of alkyl sulfates, alkyl ether sulfates, linear alpha-olefin sulfonates, dialkyl sulfosuccinates, alkylamidosulfosuccinates, and alkyl taurates each having from about C₁₂ to C₁₈ alkyl or alkenyl groups. Particular examples include the salts of lauryl sulfates and lauryl ether sulfates the latter having an average level of ethoxylation of 1-3.

Representative examples of suitable cationic surfactants include quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84.

Co-emulsifiers are typically present in compositions of the present invention in amounts ranging from about 0.1% to about 25% by weight, in some embodiments from about 0.5% to about 15%, and in some other embodiments from about 1% to about 10%, by weight, based on the total weight of the composition.

The amount of the co-emulsifier used may affect the form of the resulting cosmetic composition. For example, in embodiments of the present invention that are formulated as a liquid, the co-emulsifier may be present in amounts ranging from about 0.1% to about 5% by weight, in some embodiments from about 0.5% to about 2%, and in some other embodiments from about 0.75% to about 1.25%, by weight, based on the total weight of the composition.

In other embodiments of the present invention that are formulated as a gel or crème, the co-emulsifier may be present in amounts ranging from about 1% to about 25% by weight, in some embodiments from about 5% to about 15%, and in some other embodiments from about 7% to about 12% by weight, based on the total weight of the composition.

### Optional Ingredients

### Solvents

The cosmetic compositions of the present invention may contain a solvent. The solvent may be aqueous or non-aqueous in nature, polar or non-polar, or volatile or non-volatile.

The solvent may or may not include water. Cosmetic compositions that are washable contain water. On the other hand, water may or may not be present in waterproof cosmetic composition. Generally, water content of washable cosmetic compositions ranges from about 20 to about 80%, by weight, and in some embodiments from about 30 to about 60%, by weight, based on the total weight of the composition. In contrast, water content of waterproof cosmetic compositions generally ranges from 0 or greater than 0 to about 60%, by weight, and in some embodiments from 0 or greater than 0 to about 35%, by weight, based on the total weight of the composition. One or more water-miscible solvents (having a miscibility in water of greater than 50% by weight at about 25° C.) may also be present. Examples include lower, i.e., C1-5 monoalcohols such as ethanol and isopropanol, C2-C8 glycols containing such as propylene glycol, ethylene glycol, butylene glycol, dipropylene glycol, and pentylene glycol, C₃-C₄ ketones, and C₂-C₄ aldehydes.

Representative examples of suitable volatile solvents include non-polar volatile hydrocarbon-based oils (which as used herein, refers to oil containing only hydrogen and carbon atoms), silicone oils (optionally comprising alkyl or alkoxy groups that are pendant or at the end of a silicone chain), and fluoro oils. Representative examples of suitable hydrocarbon-based oils include isoparaffins, i.e., branched alkanes containing 8-16 carbon atoms, such as isododecane (also known as 2,2,4,4,6-pentamethylheptane), and petroleum distillates. Representative examples of suitable silicone oils may include linear or cyclic silicones containing from 2 to 7 silicon atoms, and which optionally contain alkyl or alkoxy groups containing from 1 to 10 carbon atoms. Examples include octamethylcyclotetrasiloxane, decamethylcyclopentasiloxane, hexadecamethylcyclohexasiloxane, heptamethylhexyltrisiloxane and heptamethyloctyltrisiloxane.

Representative examples of suitable polar volatile solvents include C₂ to C₅ alcohols, such as ethanol, ethyl 3-ethoxypropionate and isohexyl neopentanoate.

A volatile solvent, if present, is in amounts generally ranging from about 0.1% to about 80%, and in some embodiments, from about 5% to about 70%, and in other embodiments, from about 10% to about 60%, by weight, based on the total weight of the composition.

The compositions of the present invention may contain a non-volatile solvent or oil. Exemplary non-volatile solvents or oils include cosmetically or dermatologically acceptable and, in general, physiologically acceptable oils, such as carbon-based, hydrocarbon-based, fluoro and/or silicone oils, of mineral, animal, plant or synthetic origin, alone or as a mixture. Representative examples of suitable non-volatile solvents or oils include synthetic esters and ethers, especially of fatty acids, for instance the oils of formulae R¹COOR² and R¹OR² in which R¹ and R² represent, independently of each other, a branched or unbranched hydrocarbon-based chain containing from 3 to 30 carbon atoms, for instance purcellin oil, isononyl isononanoate, isopropyl myristate, ethylhexyl palmitate (commercially available from Croda Chemicals as Crodamol OP), 2-octyldodecyl stearate, 2-octyldodecyl erucate, isostearyl isostearate or isononyl isononanoate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate (Prisorine 3631); linear or branched hydrocarbons of mineral or synthetic origin, such as liquid paraffins, and derivatives thereof. Representative examples of silicone oils are methicone phenyl trimethicone, commercially available from Dow-Corning as DC 556, and caprylyl methicone, commercially available from Dow-Corning as FZ-3196. Further examples of non-volatile solvents or oils include polyalphaolefins, which include ethylene derivatives oligomerized into even-numbered carbon polyalphaolefins e.g., C₆-C₁₄ olefins such as polydecene and polymers of C₆, C₈, C₁₂ and C14 olefins. The polyolefins may have a molecular weight (MW) generally ranging from about 280 to about 11,500, and a viscosity (CPs at about 20° C.) generally ranging from about 7 to about 32,500. They may also be hydrogenated, for example, hydrogenated polyisobutene. In some embodiments, the non-volatile solvent includes PureSyn™ 2 (MW about 283), 4 (MW about 432), 6 (MW about 570), 8 (MW about 611), 150 (MW about 3980) and 300 (MW about 4870) (INCI name: hydrogenated polydecene). The viscosity of these polymers is about 8, about 33, about 64, about 103, about 4179 and about 8400, respectively.) PureSyn™ 100 (MW about 2939, viscosity about 3900, INCI name: hydrogenated C6-14 olefin polymers) and PureSyn™ 1000 (MW about 11,500, viscosity about 32,400, INCI name: polydecene) may also be useful. The PureSyn™ products are available from Exxon Chemicals.

A non-volatile solvent, if present, is in amounts generally ranging from about 0.1% to about 70%, and in some embodiments, about 0.5% to about 40%, and in other embodiments, 1% to about 25%, by weight, based on the total weight of the composition.

### Thickener

The cosmetic compositions of the present invention may include a thickener. The thickeners include water-soluble thickeners and oil phase thickeners. Representative examples of suitable water-soluble thickeners include polyvinylpyrrolidone (PVP), polyvinyl alcohol, crosslinked acrylates (e.g. Carbopol 982), hydrophobically-modified acrylates (e.g. Carbopol 1382); polyacrylamides such as, for example, the crosslinked copolymers sold under the names Sepigel 305 (CTFA name: polyacrylamide/C13-C14 isoparaffin/Laureth 7) or Simulgel 600 (CTFA name: acrylamide/sodium acryloyldimethyltaurate copolymer/isohexadecane/polysorbate 80) by SEPPIC; 2-acrylamido-2-methylpropanesulphonic acid polymers and copolymers, that are optionally crosslinked and/or neutralized; cellulose derivatives such as hydroxyethylcellulose, sodium carboxymethylcellulose, hydroxypropyl methylcellulose, hydroxypropyl cellulose, ethyl cellulose and hydroxymethyl cellulose; polysaccharides and gums, e.g., natural gums such as xanthan gum (e.g., Rhodicare XC from Rhodia Inc. (Cranbury, N.J.), sclerotium, carrageenan and pectin; polysaccharide resins such as starch and its derivatives, hyaluronic acid and its salts, clays, and, in particular, montmorillonites, hectorites, bentonites, and laponites, crosslinked polyacrylic acids, such as the “Carbopol” products from the company Goodrich, the polyglyceryl (meth)acrylates polymers sold under the names “Hispagel” or “Lubragel” by the companies Hispano Quimica or Guardian, crosslinked acrylamide polymers and copolymers, such as those sold under the names “PAS 5161” or “Bozepol C” by the company Hoechst, “Sepigel 305” by the company SEPPIC, crosslinked methacryloyloxyethyltrimethylammonium chloride homopolymers sold under the name “Salcare SC95” by the company Allied Colloid, and associative polymers and, in particular associative polyurethanes.

Representative examples of suitable oil phase thickeners include modified clays, such as magnesium aluminum silicate and hectorites modified with an ammonium chloride of a C₁₀ to C₂₂ fatty acid, such as hectorite modified with distearyldimethylammonium chloride, also known as quaternium-18 bentonite, such as the products sold or made under the names Bentone 34 by the company Rheox, Claytone XL, Claytone 34 and Claytone 40 sold or made by the company Southern Clay, the modified clays known under the name quaternium-18 benzalkonium bentonites and sold or made under the names Claytone HT, Claytone GR and Claytone PS by the company Southern Clay, the clays modified with stearyldimethylbenzoylammonium chloride, known as stearalkonium bentonites, such as the products sold or made under the names Claytone APA and Claytone AF by the company Southern Clay, and Baragel 24 sold or made by the company Rheox. Other mineral gelling agents include silica, such as fumed silica. The fumed silica may have a particle size ranging from about 5 nm to 200 nm.

A thickener, if present, is in amounts generally ranging from about 0.1% to about 5%, and in some embodiments, about 0.5% to about 1%, by weight, based on the total weight of the composition.

### Colorant

The inventive compositions may also contain a colorant, e.g., pigment. Pigments may be chosen from the organic and/or mineral pigments known in the art. These pigments may be in the form of pigmentary powder or paste. They may be coated or uncoated.

The pigments may be chosen, for example, from Natural (mineral) pigments, organic pigments, and pigments with special effects such as nacres or glitter flakes.

Natural pigments may be chosen from, for example, iron oxides, mica (e.g., mica sericite), chromium oxides, manganese violet, ultramarine blue, titanium dioxide, chromium hydrate and ferric blue.

The pigments may be surface-treated, i.e., have totally or partially undergone a surface treatment of chemical, electronic, electrochemical, mechanochemical or mechanical nature, with an organic agent. These organic agents may be chosen, for example, from amino acids; waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminium salts of fatty acids, for example aluminium stearate or laurate; metal alkoxides; polysaccharides, for example chitosan, cellulose and derivatives thereof; polyethylene; (meth)acrylic polymers, for example polymethyl methacrylates; polymers and copolymers containing acrylate units; proteins; alkanolamines; silicone compounds, for example silicones, polydimethylsiloxanes, alkoxysilanes, alkylsilanes and siloxysilicates; organofluorine compounds, for example perfluoroalkyl ethers; fluorosilicone compounds.

The organic pigment may be chosen from nitroso, nitro, azo, xanthene, quinoline, anthraquinone, phthalocyanin, metal-complex, isoindolinone, isoindoline, quinacridone, perinone, perylene, diketopyrrolopyrrole, thioindigo, dioxazine, triphenylmethane and quinophthalone compounds. Representative examples of suitable surfact treated pigments include perfluoroalcohol phosphate- (PF-) treated titanium dioxide and PF-treated iron oxide.

White or colored organic pigments may be chosen from carmine, carbon black, aniline black, melanin, azo yellow, quinacridone, phthalocyanin blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenolic derivatives as described in patent FR 2 679 771.

Examples that may also be mentioned include pigmentary pastes of organic pigments, such as the product sold by the company Hoechst under the names: Jaune Cosmenyl IOG: Pigment Yellow 3 (CI 11710); Jaune Cosmenyl G: Pigment Yellow 1 (CI 11680); Orange Cosmenyl GR: Pigment Orange 43 (CI 71105); Rouge Cosmenyl R: Pigment Red 4 (CI 12085); Carmine Cosmenyl FB: Pigment Red 5 (CI 12490); Violet Cosmenyl RL: Pigment Violet 23 (CI 51319); Bleu Cosmenyl A2R: Pigment Blue 15.1 (CI 74160); Vert Cosmenyl GG: Pigment Green 7 (CI 74260); Noir Cosmenyl R: Pigment Black 7 (CI 77266).

The pigments may also be in the form of composite pigments as described in EP 1 184 426. These composite pigments may be compounds of particles comprising a mineral core, at least one binder for ensuring the binding of the organic pigments to the core, and at least one organic pigment at least partially covering the core.

The organic pigment may also be a lake. The term “lake” means insolubilized dyes adsorbed onto insoluble particles, the assembly thus obtained remaining insoluble during use.

The inorganic substrates onto which the dyes are adsorbed include, for example, alumina, silica, calcium sodium borosilicate or calcium aluminium borosilicate, and aluminium.

Among the dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 0 (CI 77 002) , D&C Green 3 (CI 42 053), D&C Blue 1 (CI 42 090).

An example of a lake that may be mentioned is the product known under the following name: D&C Red 7 (CI 15 850:1).

The pigment may also be a pigment with special effects. The term “pigments with special effects” means pigments that generally create a non-uniform colored appearance (characterized by a certain shade, a certain vivacity and a certain lightness) that changes as a function of the conditions of observation (light, temperature, observation angles, etc.). They thus contrast with white or colored pigments that afford a standard uniform opaque, semi-transparent or transparent shade.

Several types of pigment with special effects exist: those with a low refractive index, such as fluorescent, photochromic or thermochromic pigments, and those with a high refractive index, such as nacres or glitter flakes.

Examples of pigments with special effects that may be mentioned include nacreous pigments such as white nacreous pigments such as mica coated with titanium or with bismuth oxychloride, colored nacreous pigments such as titanium mica with iron oxides, titanium mica with ferric blue or with chromium oxide, titanium mica with an organic pigment of the abovementioned type, and also nacreous pigments based on bismuth oxychloride. Nacreous pigments that may be mentioned include the Cellini nacres sold by Engelhard (mica-TiO₂-lake), Prestige sold by Eckart (mica-TiO₂), Prestige Bronze sold by Eckart (mica-Fe₂O₃), and Colorona sold by Merck (mica-TiO₂—Fe₂O₃).

In addition to nacres on a mica support, multilayer pigments based on synthetic substrates such as alumina, silica, sodium calcium borosilicate or calcium aluminium borosilicate, and aluminium, may be envisaged.

Mention may also be made of pigments with an interference effect that are not fixed onto a substrate, for instance liquid crystals (Helicones HC from Wacker), holographic interference flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

Quantum dots are luminescent semiconductive nanoparticles capable of emitting, under light excitation, irradiation with a wavelength ranging from 400 nm to 700 nm. These nanoparticles are known from the literature. They may be manufactured in particular according to the processes described, for example, in U.S. Pat. No. 6,225,198 or U.S. Pat. No. 5,990,479, in the publications cited therein, and also in the following publications: Dabboussi B. O. et al. “(CdSe)ZnS core-shell quantum dots: synthesis and characterization of a size series of highly luminescent nanocrystallites” Journal of Physical Chemistry B, vol. 101, 1997, pp. 9463-9475 and Peng, Xiaogang et al. “Epitaxial growth of highly luminescent CdSe/CdS core/shell nanocrystals with photostability and electronic accessibility”, Journal of the American Chemical Society, vol. 119, No. 30, pp. 7019-7029.

The variety of pigments that may be used makes it possible to obtain a wide range of colors, and also particular optical effects such as metallic effects or interference effects.

A colorant, such as a pigment, if present, is in amounts generally ranging from about 0.01% to about 50%, based on the total weight of the composition. In certain embodiments, the colorant is present in an amount of from about 0.1% to about 40%, by weight, in other embodiments from about 1% to about 20%, by weight, and in yet other embodiments from about 2% to about 10%, by weight, based on the total weight of the composition.

### Filler

The compositions of the present invention may contain a filler (e.g., lamellar or non-lamellar, inorganic or synthetic, colorless or white particles). Representative examples of suitable fillers include silica, mothers-of-pearl, boron nitride, mica, kaolin, iron oxides, titanium dioxide, polyamide powders, for instance nylon-l2 and Nylon® (Orgasol from Atochem), poly-alanine powders, polyethylene powders, tetrafluoroethylene polymer powders, for instance Teflon®, starch, hollow polymer microspheres such as those of polyvinylidene chloride/acrylonitrile, for instance Expancel® (Nobel Industrie), acrylic powders such as Polytrap® (Dow Corning), polymethyl methacrylates particles and silicone resin microbeads (for example Tospearls® from Toshiba), magnesium hydrocarbonate, hydroxyapatite, hollow silica microspheres (Silica Beads® from Maprecos), and glass and ceramic microcapsules. Mothers-of-pearl should be understood to mean iridescent particles produced especially by certain mollusks in their shell or else synthesized. Fillers may be in powderous form. A filler, if present, is in amounts generally ranging from about 0.1% to about 25%, and in some embodiments from about 1% to about 10%, by weight, based on the total weight of the composition.

### Humectant

The compositions of the present invention may contain a humectant. Representative examples of suitable humectants include glycerin, diglycerin, triglycerin, polyglycerin, ethoxylated and propoxylated glycerols polypropylene glycol, polyethylene glycol, ethylene glycol, diethylene glycol, triethylene glycol, propylene glycol, dipropylene glycol, hexylene glycol, 1,3-butylene glycol and 1,4-butylene glycol.

A humectant if present, is in amounts generally ranging from about 0.1% to about 20%, and in some embodiments, about 0.5% to about 15%, and in other embodiments, 1% to about 10%, by weight, based on the total weight of the composition.

### Preservative

The cosmetic compositions of the present invention may also contain a preservative. Representative examples of suitable preservatives include caprylyl glycol, chlorphenesin, alkyl para-hydroxybenzoates, wherein the alkyl radical has from 1, 2, 3, 4, 5 or 6 carbon atoms and preferably from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben), and phenoxyethanol. Mixtures of preservatives are commercially available, e.g., the mixture of methylparaben, ethylparaben, propylparaben and butylparaben sold under the name Nipastat by Nipa, the mixture of phenoxyethanol, methylparaben, ethylparaben, propylparaben and butylparaben, also sold by Nipa under the name Phenonip, and the mixture of phenoxyethanol, methylparaben, isopropylparaben, isobutylparaben and butylparaben, sold by ISP under the tradename Liquapar Optima. A preservative, if present, is in amounts generally ranging from about 0.01% to about 15% by weight, based on the total weight of the composition.

### Polymer

The cosmetic compositions of the present invention may also contain a polymer such as a conventional film-forming polymer. Representative examples of suitable polymers include keratin derivatives, such as keratin hydrolysates and sulphonic keratins; anionic, cationic, amphoteric or nonionic derivatives of chitin or chitosan; cellulose derivatives such as hydroxyethylcellulose, hydroxypropylcellulose, methylcellulose, ethylhydroxyethylcellulose, carboxymethylcellulose, and quaternized derivatives of cellulose; acrylic polymers or copolymers, such as polyacrylates or polymethacrylates, e.g., hydroxyethyl acrylate/sodium acryloyldimethyl taurate copolymer; polyvinylpyrrolidones (PVP) and vinyl copolymers, such as methyl vinyl ether-maleic anhydride copolymers, dimeticone/vinyl dimethicone crosspolymers, or vinyl acetate-crotonic acid copolymer; water-dispersible anionic polyesteramide and/or polyester polymers comprising monomers bearing a functional group —SO₃M, in which M represents a hydrogen atom, an ammonium ion NH₄⁺ or a metal ion, such as, for example, an Na⁺, Li⁺, K⁺, Mg²⁺, Ca²⁺, Cu²⁺, Fe²⁺ or Fe³⁺ ion; polyurethane polymers, especially anionic, cationic, nonionic or amphoteric polyurethanes, acrylic polyurethanes, polyvinylpyrrolidone polyurethanes, polyester polyurethanes, polyether polyurethanes, polyureas, polyurea/polyurethanes, and mixtures thereof; and polymers of natural origin, modified if desired, such as gum arabic, guar gum, xanthan derivatives, karaya gum; alginates and carragheenates; glycoaminoglycans, hyaluronic acid and its derivatives; shellac, sandarac gum, dammars, elemis and copals, are also useful. A polymer, if present, is in amounts generally ranging from about 0.1% to about 20% by weight, based on the total weight of the composition.

The compositions of the present invention may contain at least one further (e.g., cosmetically or dermatologically acceptable) ingredient, including additives and adjuvants, including, for example, waxes, moisturizers, gelling activators, anti-foam agents (e.g., simethicone, which is a fluid composition containing polydimethylsiloxane and silica), sunscreen agents (e.g., inorganic sunscreen agent, such as titanium dioxide and zinc oxide and organic sunscreen agents, such as octocrylene, ethylhexyl methoxycinnamate,and avobenzone), dispersion enhancing agents, fibers, chelators (such as EDTA and salts thereof, particularly sodium and potassium salts), antioxidants (e.g., BHT, tocopherol), essential oils, fragrances, and cosmetically active agents and dermatological active agents such as, for example, anti-inflammatory agents, vitamins, and trace elements. These ingredients may be soluble or dispersible in whatever phase or phases is/are present in the cosmetic composition (i.e., aqueous and/or fatty phase).

The compositions for application to keratinous tissue may constitute in particular a cosmetic or dermatological composition for protecting, treating, or caring for keratinous tissue, such as skin, hair, including eyelashes and eyebrows, and lips. In certain embodiments, the cosmetic compositions of the present invention are in the form of a foundation, an eye shadow, mascara, an eyeliner, a blusher, a lipstick, a lipgloss, a care base or balm for the lips, or a concealer product. The cosmetic compositions of the present invention may be applied to keratinous tissue by hand. Alternatively, or in conjunction therewith, they may be applied via an applicator such as a sponge, cotton, brush, or a puff of a natural or synthetic material. In addition, the applicator may be attached to or contained within a container that serves as a reservoir for the cosmetic composition.

It has been surprisingly discovered that a composition according to an embodiment of the present invention has excellent physical properties and good sheen, without any of the drawbacks of conventional neutralizers. Emulsions employing arginine as a neutralizer are soft creams with good sheen and no unpleasant odor. Moreover, arginine is one of the essential amino acids produced by the human body and is an essential building block of the proteins that are components of the skin. Arginine plays an important role in cell division and helps with the healing of wounds. Thus, the compositions of the present invention achieve excellent results and also employ a neutralizer commonly found in and beneficial to the body.

The following examples are intended to further illustrate the present invention. They are not intended to limit the invention in any way. Unless otherwise indicated, all parts are by weight.

### EXAMPLES

### Example 1

A foundation composition of the present invention is described below.

| PHASE | INGREDIENTS | AMOUNT (Wt %) |
|---|---|---|
| A1 | Water | 58.89 |
| | Xanthan Gum | 0.30 |
| | Magnesium Aluminum | 0.25 |
| | Silicate | |
| | Sodium Stearoyl | 0.50 |
| | Lactylate | |
| | Caprylyl Glycol | 0.50 |
| | Glycerin | 5.00 |
| A2 | Titanium Dioxide | 8.04 |
| | Iron Oxides | 1.96 |
| A3 | Arginine | 0.21 |
| B | Ethylhexyl Palmitate | 11.00 |
| | Dicapryl Ether | 8.00 |
| | Cetyl Alcohol | 0.50 |
| | Stearic Acid | 1.85 |
| C | Silica | 2.00 |
| D | Mica | 1.00 |
| | | 100.00 |

The foundation composition described above was prepared as follows. The ingredients of Phases A1 and A3 were added to a main beaker and ground. The ingredients of Phase A2 were pre-mixed and added to the main beaker. The mixture of Phases A1-A3 was then ground for 20-25 minutes under the Silverson® grinder at about 5,000 to 6,000 rpm. The mixture was transferred to the Rayneri® mixer and mixed until uniform with heating to 60-65° C. In a separate beaker the ingredients of Phase B were heated to 60-65° C. and mixed until all were melted and uniform. When both mixtures were at a temperature between 60 and 65° C., the Phase B ingredients were added to the main beaker and homogenized for 10-12 minutes. The mixture was then transferred to a Caframo® mixer and mixed under a sweep/paddle blade on low speed while cooling to 25° C. At 25° C., the Phase C ingredient (silica) was added and mixed until uniform. The ingredient of Phase D (mica) was then added and mixed until uniform.

### Example 2

| PHASE | INGREDIENTS | AMOUNT (Wt %) |
|---|---|---|
| A1 | Water | 42.289 |
| | Glycerin | 5.00 |
| | Butylene Glycol | 4.00 |
| | Chlorphenesin | 0.20 |
| | Disodium EDTA | 0.05 |
| | Methylparaben | 0.20 |
| | Propylparaben | 0.15 |
| | Phenoxyethanol | 0.80 |
| | Magnesium Aluminum Silicate | 0.30 |
| A2 | Hydroxyethyl Acrylate/Sodium | 1.10 |
| | Acryloyldimethyl | |
| | Taurate Copolymer | |
| A3 | Arginine | 0.67 |
| B1 | Octocrylene | 4.00 |
| | Ethylhexyl Methoxycinnamate | 7.50 |
| | Ethylhexyl Palmitate | 10.00 |
| | BHT | 0.09 |
| | Butylparaben | 0.15 |
| | PF-Treated Titanium Dioxide | 3.28 |
| | PF-Treated Iron Oxide | 0.72 |
| B2 | Stearic Acid | 1.85 |
| | Glyceryl Stearate | 1.05 |
| | PEG-100 Stearate | 1.80 |
| | and Glyceryl Stearate | |
| | Cetyl Alcohol | 1.55 |
| B3 | Dimethicone/Vinyl | 1.50 |
| | Dimethicone Crosspolymer | |
| | (DC 9701 Cosmetic | |
| | Powder from Dow Corning) | |
| | Cyclopentasiloxane | 5.00 |
| C | Petrolatum and Ceteth-10 and Steareth-12 | 5.00 |
| | and Poloxamer 335 emulsion | |
| | (DC 7-3105 Petrolatum HIP Emulsion | |
| | from Dow Corning) | |
| | Mica and Titanium Dioxide | 1.75 |
| | | 100.00 |

The foundation composition described above was prepared as follows. The ingredients of Phase A1 were added to a main kettle and heated to 60-65° C. and mixed until all components were dissolved and uniform. The Phase A2 ingredients were added to the main kettle and mixed until completely hydrated. The Phase A3 ingredients were added to the main kettle and mixed until uniform. In a separate container the ingredients of Phase B1 were ground under a Silverson® grinder for 20 minutes and then switched to a regular mixer and heated to 60° C. In another beaker the ingredients of Phase B2 were completely melted and added to the ingredients of Phase B1. This mixture was then mixed until the ingredients were completely dissolved and uniform and heated to 60° C. The ingredients of Phase B3 were then added and mixed until the ingredients were completely dissolved and uniform. When the Phase A1-A3 and Phase B1-B3 mixtures are both at 60° C., the Phase B1-B3 mixture is added to the Phase A1-A3 mixture to form an emulsion and mixed for 10-15 minutes. The mixture was then cooled to 40° C. and Phase C was added and mixed until uniform.

### Example 3

| PHASE | INGREDIENTS | AMOUNT (Wt %) |
|---|---|---|
| A1 | Water | 42.059 |
| | Glycerin | 5.00 |
| | Butylene Glycol | 4.00 |
| | Chlorphenesin | 0.20 |
| | Disodium EDTA | 0.05 |
| | Methylparaben | 0.20 |
| | Propylparaben | 0.15 |
| | Phenoxyethanol | 0.80 |
| | Magnesium Aluminum Silicate | 0.30 |
| A2 | Hydroxyethyl Acrylate/Sodium | 1.10 |
| | Acryloyldimethyl Taurate | |
| | Copolymer | |
| A3 | Arginine | 0.90 |
| B1 | Octocrylene | 4.00 |
| | Ethylhexyl Methoxycinnamate | 7.50 |
| | Ethylhexyl Palmitate | 10.00 |
| | BHT | 0.09 |
| | Butylparaben | 0.15 |
| | PF-Treated Titanium Dioxide | 3.28 |
| | PF-Treated Iron Oxide | 0.72 |
| B2 | Stearic Acid | 1.85 |
| | Glyceryl Stearate | 1.05 |
| | PEG-100 Stearate and Glyceryl | 1.80 |
| | Stearate | |
| | Cetyl Alcohol | 1.55 |
| B3 | Dimethicone/Vinyl | 1.50 |
| | Dimethicone Crosspolymer (DC | |
| | 9701 Cosmetic Powder from Dow | |
| | Corning) | |
| | Cyclopentasiloxane | 5.00 |
| C | Petrolatum and Ceteth-10 and | 5.00 |
| | Steareth-12 and Poloxamer 335 | |
| | emulsion (DC 7-3105 Petrolatum HIP | |
| | Emulsion from Dow Corning) | |
| | Mica and Titanium Dioxide | 1.75 |
| | | 100.00 |

The foundation composition described above was prepared as described in Example 2.

All publications cited in the specification, both patent publications and non-patent publications, are indicative of the level of skill of those skilled in the art to which this invention pertains. All these publications are herein incorporated by reference to the same extent as if each individual publication were specifically and individually indicated as being incorporated by reference. In addition, commonly owned application Ser. No. 13/078,418, entitled “Cosmetic Compositions Containing Arginine and a Preservative System Containing an Alcohol,” filed of even date herewith, and which claims the benefit of the filing date of U.S. Provisional Application No. 61/320,028, is hereby incorporated by reference in its entirety.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic composition comprising:
a) a fatty acid;
b) arginine; and
c) a co-emusifier.

2. The cosmetic composition of claim 1, wherein the fatty acid is a C₁₂-C₁₆ fatty acid.

3. The cosmetic composition of claim 2, wherein the C₁₂-C₁₆ fatty acid is stearic acid.

4. The cosmetic composition of claim 1, wherein the co-emulsifier is selected from nonionic surfactants, anionic surfactants, and cationic surfactants.

5. The cosmetic composition of claim 4, wherein the co-emulsifier is a fatty alcohol.

6. The cosmetic composition of claim 1, wherein the coemulsifier is cetyl alcohol and sodium stearoyl lactylate.

7. The cosmetic composition of claim 1, further comprising a solvent, a thickener, a colorant, a filler, a humectant, and a preservative.

8. The cosmetic composition of claim 7, wherein the solvent is selected from water, ethylhexyl palmitate, and dicapryl ether.

9. The cosmetic composition of claim 7, wherein the thickener selected from xantham gum and magnesium aluminum silicate.

10. The cosmetic composition of claim 7, wherein the colorant is iron oxide.

11. The cosmetic composition of claim 7, wherein the filler is selected from mica and silica.

12. The cosmetic composition of claim 7, wherein the humectant is glycerin.

13. The cosmetic composition of claim 7, wherein the preservative is selected from the group consisting of caprylyl glycol, methylparaben, propylparaben, and phenoxyethanol.

14. The cosmetic composition of claim 1, comprising stearic acid, arginine, cetyl alcohol, sodium stearoyl lactylate, a solvent, a thickener, a colorant, a filler, a humectant, and a preservative.

15. A process for making a cosmetic composition comprising mixing together:
a) a fatty acid,
b) arginine, and
c) a co-emulsifier.

16. The process of claim 15, wherein the fatty acid is a C₁₂-C₁₆ fatty acid.

17. The process of claim 16, wherein the C₁₂-C₁₆ fatty acid is stearic acid.

18. The process of claim 15, wherein the co-emulsifier is cetyl alcohol and sodium stearoyl lactylate.

19. A method of making up or enhancing the appearance of a keratinous tissue comprising applying to the keratinous tissue a cosmetic composition comprising:
a) a fatty acid;
b) arginine; and
c) a co-emusifier.
